(19) United States
(12) Patent Application Publication
(10) Pub. No.: US 2025/0057746 A1
(43) Pub. Date: Feb. 20, 2025

(54) OIL-IN-WATER TYPE D-PHASE EMULSION COMPOSITION WITH EXCELLENT EMULSION STABILITY

(71) Applicant: LG H&H CO., LTD., Seoul (KR)

(72) Inventors: Na Eun YOOK, Seoul (KR); Hyun Min HWANG, Seoul (KR)

(73) Assignee: LG H&H CO., LTD., Seoul (KR)

(21) Appl. No.: 18/723,232

(22) PCT Filed: Dec. 22, 2022
(86) PCT No.: PCT/KR2022/021063

§ 371 Date: Jun. 21, 2024

(30) Foreign Application Priority Data
Dec. 23, 2021 (KR) 10-2021-0185858

(51) Int. Cl. A61K 8/44; A61Q 19/00; A61Q 19/08

(52) U.S. Cl. CPC A61K 8/442; A61Q 19/007; A61Q 19/08; A61K 2800/52

(57) ABSTRACT

The present invention relates to an oil-in-water D-phase emulsion composition, which includes: N-acyl proline or a salt thereof; and an oil, and a method of preparing the same. The composition according to the present invention can exhibit low viscosity while containing a high oil content, can provide excellent emulsion stability, and also allows for high moisturizing properties and a moist and soft sensation of use.

### TECHNICAL FIELD

The present invention relates to a composition having excellent emulsion stability while including a high content of oil, and specifically, to an oil-in-water D-phase emulsion composition including N-acyl proline or a salt thereof and an oil.

### BACKGROUND ART

In order to maintain healthy skin, it is necessary to provide adequate moisture and oil to the skin. Cosmetics are used as a means for protecting the skin from the external environment, recovering the skin and maintaining its health. Also, cosmetics have the function of replenishing the skin's moisture and oil lost due to changes in the external environment and delaying skin aging.

Among cosmetics, skin toners are able to be more easily applied to the skin than lotions or creams, provide a refreshing feeling of use, and enable immediate moisture supply to the skin, but they have difficulty in maintaining skin moisture for a long period of time. When skin toners are mixed with fat-soluble oil-phase ingredients to allow the skin to maintain an appropriate oil-water balance, the ingredients cannot be mixed in a large amount due to stability issues such as separation and the like. Therefore, to mix with a large amount of fat-soluble ingredients, it is common to use an emulsifier for maintaining the stability of oil emulsion particles. However, since most emulsifiers are solids, when a high content of oil is emulsified, the viscosity or hardness of a composition is increased due to the used emulsifier itself, and as a result, only high-viscosity formulations such as lotions or creams can be made.

Although oil-in-water formulations using amphiphilic polymers without using solid emulsifiers have been developed, this method has the problem in that due to the nature of the polymer having relatively low emulsifying activity, as the oil content increases, the polymer content also increases for stability, and as a result, high-viscosity formulations are inevitably formed, which makes it difficult to realize a refreshing feeling of use like skin toners.

In addition, there is a known technology for stabilizing a high content of oil in low-viscosity formulations using special equipment such as a high-pressure emulsification device, but this emulsification method has the limitation of being complicated and expensive.

Therefore, there is still a need for a low-viscosity stable oil-in-water formulation that can be simply prepared without using special equipment while including a high content of oil.

### DISCLOSURE

### Technical Problem

The inventors of the present invention have found that, when the amino acid corresponding to the hydrophilic portion in lipoamino acids having an amphiphilic structure is proline, a strong hydrophilic portion is formed, and thus it is advantageous for D-phase emulsification, thereby allowing a high content of oil to be stably emulsified with low viscosity.

Therefore, the present invention is directed to providing an oil-in-water D-phase emulsion composition, which has high emulsion stability and exhibits low viscosity while including a high content of oil.

### Technical Solution

One aspect of the present invention provides an oil-in-water D-phase emulsion composition including: N-acyl proline or a salt thereof; and an oil.

Another aspect of the present invention provides a method of preparing an oil-in-water D-phase emulsion composition, which includes: preparing a D-phase including N-acyl proline or a salt thereof; mixing the D-phase with an oil phase including an oil; and mixing the mixture with an aqueous phase.

### Advantageous Effects

According to the present invention, an oil-in-water composition, which has low viscosity and excellent emulsion stability while including a large amount of oil, can be provided by using N-acyl proline or a salt thereof as an emulsifier for D-phase emulsification. Also, since such a composition has excellent moisturizing properties and is capable of providing a moist and emollient feeling of use, it can secure high value as a cosmetic with skin improvement effects while simultaneously increasing emulsion stability.

### DESCRIPTION OF DRAWINGS

FIG. 1 is a schematic diagram showing a method of preparing an oil-in-water D-phase emulsion composition according to the present invention.

FIG. 2 is a graph showing a skin brightness change according to Experimental Example 2.

FIG. 3 a graph showing a skin redness change according to Experimental Example 2.

FIG. 4 shows images for confirming formulation stability over time according to Experimental Example 4.

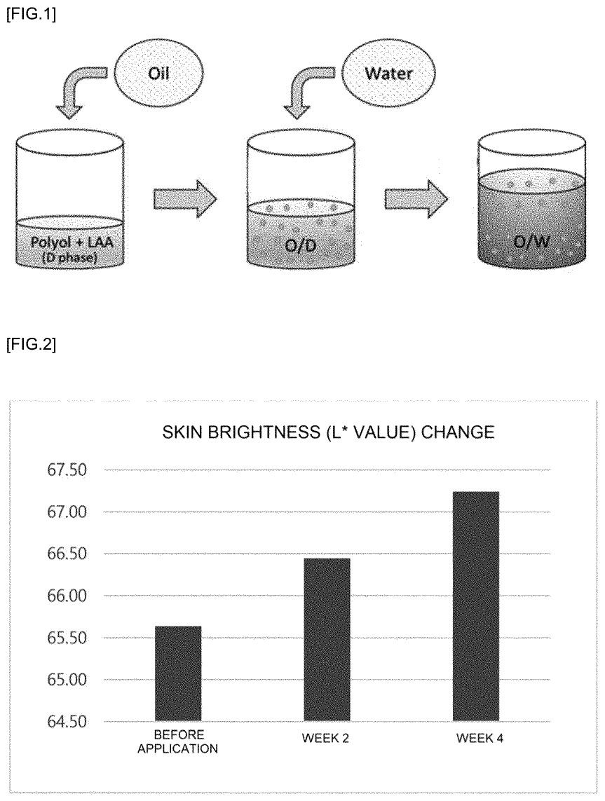

### MODES OF THE INVENTION

The present invention relates to an oil-in-water D-phase emulsion composition including: N-acyl proline or a salt thereof; and an oil.

Lipoamino acids having an amphiphilic structure include a hydrophilic portion corresponding to an amino acid and a hydrophobic portion corresponding to a hydrocarbon residue. The lipoamino acid may be expressed as an N-acyl amino acid, and since proline is relatively polar among amino acids, N-acyl amino acid with proline as the hydrophilic portion is capable of forming a strong hydrophilic portion, and thus is highly advantageous in lowering the interfacial energy between an oil phase and an aqueous phase. Also, since N-acyl proline or a salt thereof is capable of stabilizing an oil phase without using other solid or paste-type emulsifiers and emulsification aids, the viscosity of the composition can be maintained at a low level.

Therefore, when including N-acyl proline or a salt thereof, the oil-in-water D-phase emulsion composition can have low viscosity and exhibit excellent emulsion stability while including a large amount of oil.

In this specification, “D-phase emulsion” refers to a product prepared by D-phase emulsification. Specifically, it means an oil-in-water (O/W) emulsion prepared by slowly adding oil to a D-phase to which an emulsifier has been added, mixing them to form an oil-in-D phase (O/D) emulsion, and adding an aqueous phase to the emulsion.

In this specification, “being stable” means that there is no phase separation or no change in appearance for a long period of time under various temperature conditions after preparation. For example, it means that the composition does not substantially separate into phases or has no substantial changes in appearance and viscosity even when stored at 20° C. to 60° C., for example, 25° C. to 50° C., for 4 weeks or more, or 4 weeks to 12 weeks, for example, 4 weeks, 8 weeks or 12 weeks or even when stored for three freeze-thaw (F/T) cycles.

In the “freeze-thaw (F/T) cycle” of this specification, “freeze” means that the composition is stored at −15° C. until it is completely frozen, and “thaw” means that the composition is stored at room temperature until it is completely thawed. In other words, “freeze-thaw (F/T) cycle” means repeatedly performing the freeze and the thaw, and for example, “three F/T cycles” means repeatedly performing the freeze and the thaw three times.

“N-acyl proline” has a structure in which the amine group of proline is substituted with an acyl group. The acyl group is —C(═O)—R, wherein R may be a saturated or unsaturated branched hydrocarbon-based chain. Specifically, R may be a C₁₂₋₂₄ alkyl, preferably a C₁₂₋₂₂ alkyl or a C₁₄₋₂₂ alkyl, and more preferably a C₁₆₋₂₀ alkyl.

In addition, “N-acyl” may be one or more selected from the group consisting of lauroyl, myristoyl, behenoyl, palmitoyl, stearoyl, isostearoyl, olivoyl, cocoyl, and oleoyl.

The specific structure of N-acyl proline is represented as follows:

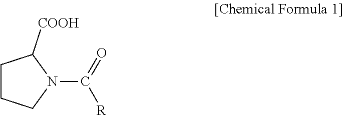

in Chemical Formula 1, R is the same as defined above.

In addition, N-acyl proline of the present invention may be present in a salt form. Here, the salt of N-acyl proline is in the form in which the —COOH group of proline is ionized into —COO⁻. The cation of the N-acyl proline salt may be an alkali metal cation such as sodium, lithium or potassium, an alkaline earth metal cation such as magnesium or calcium, or an ammonium cation, but the present invention is not limited thereto.

Preferred examples of N-acyl proline or a salt thereof include palmitoyl proline, sodium palmitoyl proline, or a mixture thereof.

The content of the N-acyl proline or salt thereof according to the present invention may be 0.5 to 5 wt %, and preferably, 0.5 to 3 wt % relative to the total weight of the composition. In the present invention, when the content of N-acyl proline or a salt thereof as an emulsifier falls within the above range, emulsification can most favorably occur, and when the content is less than the above range, emulsifying activity may not be sufficient to emulsify a high content of oil, and thus emulsion stability may be degraded.

The specific type of oil that can be stabilized in the present invention is not particularly limited as long as it is typically used in an oil-in-water emulsion composition. For example, the oil may include one or more selected from the group consisting of a silicone oil, an ester-based oil, a hydrocarbon-based oil, a triglyceride-based oil and a mixture thereof.

The silicone oil may be one or more selected from the group consisting of dimethicone, cyclomethicone, polydimethylsiloxane, methylphenylpolysiloxane, methylcyclopolysiloxane, octamethylcyclotetrasiloxane, decamethylcyclopentasiloxane, dodecamethylcyclohexasiloxane, tetradecamethylhexasiloxane, and octamethyltrisiloxane, but present invention is not limited thereto.

The ester-based oil may be one or more selected from the group consisting of triethylhexanoin, coco-caprylate/caprate, cetyl ethylhexanoate, cetyl octanoate, cetyl isooctanoate, octyldodecyl myristate, pentaerythrityl tetraethylhexanoate, isopropyl palmitate, isopropyl myristate, caprylic/capric triglyceride, butylene glycol dicaprylate/dicaprate, tocopherol acetate, and dicaprylic carbonate, but present invention is not limited thereto.

The hydrocarbon-based oil may be one or more selected from the group consisting of paraffin, ceresin and microcrystalline waxes, C₁₈₋₂₁ alkanes, isohexadecane, isododecane, undecane, squalane, squalene, hydrogenated polydecene, and hydrogenated polyisobutene, but present invention is not limited thereto.

The triglyceride-based oil may be one or more selected from the group consisting of C₈₋₁₂ acid triglyceride, C₁₂₋₁₈ acid triglyceride, caprylic/capric triglyceride, caprylic/capric/lauric triglyceride, C₁₀₋₄₀ isoalkyl acid triglyceride, C₁₀₋₁₈ triglyceride, glyceryl triacetyl hydroxystearate, soybean glyceride, tribehenin, tricaprin, triethylhexanoin, triheptanoin, triisostearin, tripalmitin, and tristearin, but present invention is not limited thereto.

The oil may preferably include one or more selected from the group consisting of caprylic/capric triglyceride, coco-caprylate/caprate, triethylhexanoin, and hydrogenated polydecene.

The content of the oil may be 5 to 40 wt %, preferably 5 to 35 wt %, and more preferably 10 to 35 wt % relative to the total weight of the composition. When the oil content is less than 5 wt %, moisturizing properties and an emollient feeling of use may be degraded, and when the oil content exceeds 40 wt %, emulsion stability may be degraded due to an inappropriate mixing ratio of a D-phase and an aqueous phase.

As D-phase emulsification is able to utilize the dispersion energy of the aqueous phase, the effects of increasing emulsion stability and lowering viscosity according to the present invention, which adds N-acyl proline or a salt thereof in the composition, may be further improved. The composition of the present invention may exhibit low viscosity. Specifically, the composition may have a viscosity of 4000 cps or less, and more specifically, 3500 cps or less, 3000 cps or less, or 2500 cps or less.

Here, viscosity refers to the resistance of a fluid to flow and is a value measured at 25° C. and 12 rpm using a Brookfield viscometer with an LV #3 (63) spindle after 1-minute stabilization. Viscosity may be measured in any other units using any other methods known in the art.

Generally, viscosity may be increased by a solid emulsifier or emulsification aid for emulsifying an oil-phase ingredient such as an oil. However, in the case of the composition of the present invention, D-phase emulsification using N-acyl proline or a salt thereof is capable of emulsifying a high content of oil, and as a result, low viscosity may be exhibited. The composition of the present invention may include a trace amount of a solid emulsifier or a solid emulsification aid such as wax or paste, or may not substantially include a solid emulsifier or a solid emulsification aid.

Specifically, the composition of the present invention may include a solid emulsifier or a solid emulsification aid in an amount of 5 wt % or less, 3 wt % or less, 1 wt % or less, 0.5 wt % or less, 0.1 wt % or less, or 0.01 wt % or less relative to the total weight of the composition, and may include almost no solid emulsifier or solid emulsification aid.

The composition of the present invention may be stable even when stored at 20 to 60° C., and specifically, 20 to 30° C. or 40 to 60° C. for 8 weeks or more. More specifically, the composition of the present invention may be stable even when stored at 25° C. for 8 weeks or more.

In particular, the composition of the present invention may be stable when stored at the above temperature for 8 weeks or more even when having a high oil content of 5 to 40 wt % or not including a solid emulsifier or emulsification aid.

The composition of the present invention may further include a polyol or a polyol derivative.

In the composition of the present invention, the polyol or polyol derivative may be mixed with N-acyl proline or a salt thereof to form a D-phase.

The polyol may include one or more selected from the group consisting of glycerin, polyhydric alcohols such as dihydric or higher alcohols, and a mixture thereof. Preferably, the polyol may include one or more selected from the group consisting of glycerin, butylene glycol, dipropylene glycol, propanediol, and 1,2-hexanediol, but the present invention is not limited thereto.

The content of the polyol may be 2 to 30 wt %, preferably 3 to 25 wt % or 5 to 25 wt %, and more preferably 10 to 25 wt % relative to the total weight of the composition.

The composition of the present invention may be in the form of a liquid or cream at room temperature.

The ingredients included in the composition of the present invention are active ingredients, and in addition to the active ingredients, ingredients typically used in a cosmetic composition may be included. For example, typical carriers or adjuvants such as preservatives, antioxidants, thickeners, stabilizers, solubilizers, vitamins, pigments, and fragrances may be included.

The composition of the present invention may be suitably used for an external preparation for skin, it is also possible to apply the composition to cosmetics, quasi-drugs, and pharmaceuticals, and may contain necessary ingredients according to each area of application. Particularly, it is preferable that the composition is applied to cosmetics such as skincare cosmetics.

The composition of the present invention may be prepared in any formulation typically prepared in the art and may be, for example, formulated into a solution, a suspension, an emulsion, a paste, a gel, a cream, a lotion, a surfactant-containing cleanser, oil, an emulsion foundation, a wax foundation, a pack, a massage cream, a spray, or the like, but the present invention is not limited thereto. More specifically, the composition may be formulated as a skin toner, a gel, a milk, a lotion, a cleansing lotion, a cleansing milk, a make-up base, or a foundation.

In addition, the present invention provides a method of preparing an oil-in-water D-phase emulsion composition including N-acyl proline or a salt thereof.

Specifically, the method of preparing an oil-in-water D-phase emulsion composition according to the present invention includes: preparing a D-phase including N-acyl proline or a salt thereof; mixing the D-phase with an oil phase including an oil; and mixing the mixture with an aqueous phase.

The content of the oil may be 5 to 40 wt % relative to the total weight of the composition.

The D-phase may include a polyol or a polyol derivative in addition to N-acyl proline or a salt thereof.

The mixing of the D-phase with the oil phase may be performed at 40 to 60° C.

In addition, the mixing of the D-phase with the oil phase may include performing stirring for 1 to 20 minutes, and preferably, 3 to 10 minutes.

The ingredients described in the preparation method may be the same as described for the composition.

The above-described ingredients included in the cosmetic composition according to the present invention may be included in the cosmetic composition of the present invention within a range not exceeding the maximum amount specified in standards related to “Cosmetic Use and Permission” prescribed by each government.

Hereinafter, the present invention will be described in detail with reference to the following experimental examples. However, it should be understood that the following experimental examples are given for the purpose of illustration only and are not intended to limit the scope of the present invention. In addition, these experimental examples are only intended to aid in understanding the present invention, and the scope of the present invention is not limited to the following experimental examples.

### EXAMPLES

### Preparation Example 1—Preparation of Oil-In-Water Emulsion Composition According to Type of Lipoamino Acid (LAA)

The raw materials of each of Part A (D-phase) and Part B (oil phase) of Table 1 below were mixed and stirred at 50° C. to prepare homogeneous mixtures. The raw materials of Part C (aqueous phase) were mixed and stirred at room temperature for 30 minutes, and thereby homogeneous mixtures of Part A, Part B, and Part C were prepared.

Part B was added to Part A while stirring Part A, and stirred using a homo mixer for 5 minutes. Part C was slowly added to the mixture of Part A and Part B while stirring the mixture for 5 minutes, stirred using a homo mixer for 5 minutes, and thereby oil-in-water compositions of Example 1 and Comparative Examples 1 to 4 were prepared with the compositions shown in the following Table 1.

TABLE 1

| | | | Comparative | Comparative | Comparative | Comparative |
|---|---|---|---|---|---|---|
| | | Example 1 | Example 1 | Example 2 | Example 3 | Example 4 |
| A | Sodium palmitoyl | 1 | — | — | — | — |
| | proline | | | | | |
| | Capryloyl glycine | — | 1 | — | — | — |
| | Undecylenoyl glycine | — | — | 1 | — | — |
| | Palmitoyl glycine | — | — | — | 1 | — |
| | Undecylenoyl | — | — | — | — | 1 |
| | phenylalanine | | | | | |
| | Glycerin | 9 | 9 | 9 | 9 | 9 |
| | Butylene glycol | 3 | 3 | 3 | 3 | 3 |
| | 1,2-Hexanediol | 1.5 | 1.5 | 1.5 | 1.5 | 1.5 |
| B | Caprylic/capric | 15 | 15 | 15 | 15 | 15 |
| | triglyceride | | | | | |
| C | Water | To 100 | To 100 | To 100 | To 100 | To 100 |
| | Carbomer | 0.1 | 0.1 | 0.1 | 0.1 | 0.1 |
| | pH adjusting agent | 0.08 | 0.08 | 0.08 | 0.08 | 0.08 |

### Experimental Example 1—Evaluation of Emulsion Stability and Viscosity

The formulation stability over time of the compositions of Example 1 and Comparative Examples 1 to 4, which were prepared according to Preparation Example 1, was observed. Specifically, whether the composition was separated or not while being stored in chambers set at 50° C. and 25° C. for 8 weeks was observed, and a freeze-thaw (F/T) cycle was repeated three times to determine freezing stability. The results thereof are shown in Table 2 below.

In addition, the viscosity of the compositions prepared according to Preparation Example 1 was measured. Specifically, the viscosity was measured at 25° C. and 12 rpm using a Brookfield viscometer with an LV #3 (63) spindle after 1-minute stabilization.

TABLE 2

| | | Comparative | Comparative | Comparative | Comparative |
|---|---|---|---|---|---|
| | Example 1 | Example 1 | Example 2 | Example 3 | Example 4 |
| Three F/T cycles | stable | precipitated, | precipitated, | precipitated, | precipitated, |
| | | separated | separated | separated | separated |
| 50° C., 8 weeks | stable | separated | separated | separated | separated |
| | | immediately after | immediately after | after 2 days | immediately after |
| | | preparation | preparation | | preparation |
| 25° C., 8 weeks | stable | separated | separated | separated | separated |
| | | immediately after | immediately after | after 10 days | immediately after |
| | | preparation | preparation | | preparation |
| Viscosity (Day 1) | 1000 cps | — | — | 1200 cps | — |

As shown in Table 2, it can be confirmed that the composition of Example 1 according to the present invention exhibited excellent formulation stability over time under all conditions, whereas the compositions of Comparative Examples 1 to 4 exhibited degraded emulsion stability and poor emulsion stability because the aqueous phase and the oil phase were separated or precipitated immediately after preparation of the composition or separation or precipitation occurred over time.

In addition, as a result of measuring the viscosity in the case of Example 1, it can be confirmed that the composition had a low viscosity of 1000 cps.

### Experimental Example 2—Evaluation of Skin Tone Improvement

In order to determine the skin tone improvement effect of the composition of Example 1 which was prepared according to Preparation Example 1, evaluation was performed as follows. 8 subjects were asked to apply the composition of Example 1 on their facial skin every morning and evening, and evaluation was performed before and 2 and 4 weeks after use of the product.

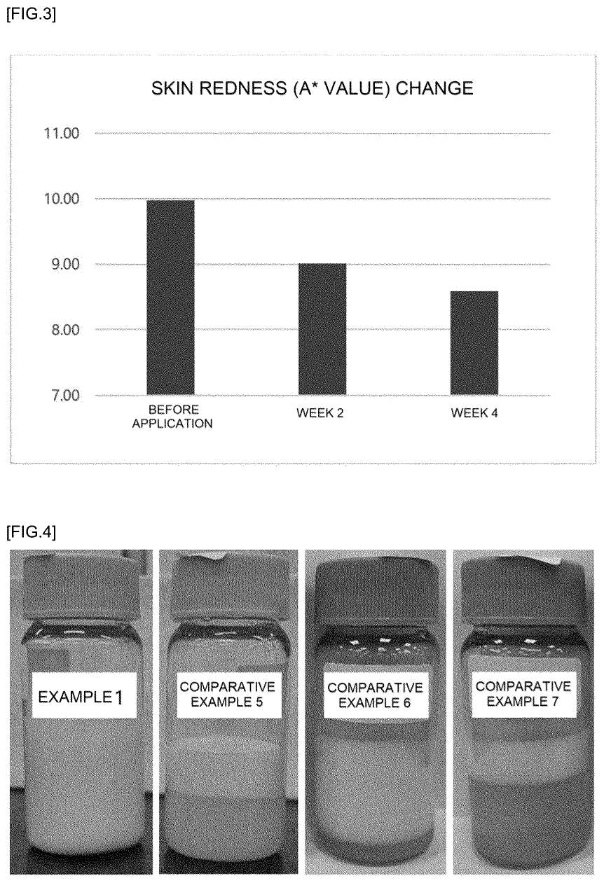

In this case, skin brightness was measured by repeatedly measuring the L* (brightness) and a* (redness) of the cheek area of the face three times using a chromameter CR-400 (Konica Minolta, Inc., Japan) after the face was washed and then rested for 20 minutes, and measurement results are shown in FIGS. 2 and 3.

As shown in FIGS. 2 and 3, it can be confirmed that the skin brightness value was increased (65.64→67.24) and the redness value was decreased (9.97→8.59) after the composition according to Example 1 of the present invention was applied for 4 weeks compared to before application. In other words, it can be seen that the composition prepared using sodium palmitoyl proline, which has brightening and soothing efficacy, as an emulsifier exhibits not only high emulsion stability at low viscosity but also excellent skin improvement effects.

### Preparation Example 2—Preparation of Oil-In-Water Emulsion Composition According to Type of Oil

The raw materials of each of Part A (D-phase) and Part B (oil phase) of Table 3 below were mixed and stirred at 50° C. to prepare homogeneous mixtures. The raw materials of Part C (aqueous phase) were mixed and stirred at room temperature for 30 minutes, and thereby homogeneous mixtures of Part A, Part B, and Part C were prepared.

Part B was added to Part A while stirring Part A, and stirred using a homo mixer for 5 minutes. Part C was slowly added to the mixture of Part A and Part B while stirring the mixture for 5 minutes, stirred using a homo mixer for 5 minutes, and thereby oil-in-water compositions of Examples 1 to 5 were prepared with the compositions shown in the following Table 3.

TABLE 3

| | | Example 1 | Example 2 | Example 3 | Example 4 | Example 5 |
|---|---|---|---|---|---|---|
| A | Sodium palmitoyl proline | 1 | 1 | 1 | 1 | 2 |
| | Glycerin | 9 | 9 | 9 | 9 | 15 |
| | Butylene glycol | 3 | 3 | 3 | 3 | 6 |
| | 1,2-Hexanediol | 1.5 | 1.5 | 1.5 | 1.5 | 1.5 |
| B | Caprylic/capric triglyceride | 15 | — | — | — | 15 |
| | Coco-caprylate/caprate | — | 15 | — | — | 6 |
| | Triethylhexanoin | — | — | 15 | — | 6 |
| | Hydrogenated polydecene | — | — | — | 15 | 3 |
| C | Water | To 100 | To 100 | To 100 | To 100 | To 100 |
| | Carbomer | 0.1 | 0.1 | 0.1 | 0.1 | 0.1 |
| | pH adjusting agent | 0.08 | 0.08 | 0.08 | 0.08 | 0.08 |

### Experimental Example 3—Evaluation of Emulsion Stability and Viscosity

The formulation stability over time and viscosity of the compositions of Examples 1 to 5, which were prepared according to Preparation Example 2, were determined in the same manner as in Experimental Example 1, and the results thereof are shown in the following Table 4.

TABLE 4

| | Example 1 | Example 2 | Example 3 | Example 4 | Example 5 |
|---|---|---|---|---|---|
| Three F/T | stable | stable | stable | stable | stable |
| cycles | | | | | |
| 50° C., | stable | stable | stable | stable | stable |
| 8 weeks | | | | | |
| 25° C., | stable | stable | stable | stable | stable |
| 8 weeks | | | | | |
| Viscosity | 1000 cps | 800 cps | 800 cps | 1000 cps | 1900 cps |
| (Day 1) | | | | | |

As shown in Table 4, it can be confirmed that the compositions according to the present invention exhibited excellent formulation stability over time and low viscosity under all conditions regardless of the type of oil.

### Preparation Example 3—Preparation of Oil-In-Water Emulsion Composition According to Amino Acid-Based Emulsifier and Emulsification Method

Oil-in-water compositions were prepared with the compositions shown in Table 5 below according to the following method.

### D-Phase Emulsification Method and Preparation—Example 1 and Comparative Example 6

The raw materials of each of Part A (D-phase) and Part B (oil phase) of Table 5 below were mixed and stirred at 50° C. to prepare homogeneous mixtures. The raw materials of Part C (aqueous phase) were mixed while stirring at room temperature for 30 minutes, and thereby homogeneous mixtures of Part A, Part B, and Part C were prepared.

Part B was added to Part A while stirring Part A, and stirred using a homo mixer for 5 minutes. Part C was slowly added to the mixture of Part A and Part B while stirring the mixture for 5 minutes, stirred using a homo mixer for 5 minutes, and thereby oil-in-water compositions of Example 1 and Comparative Example 6 were prepared with the compositions shown in the following Table 5.

### General Emulsification Method and Preparation—Comparative Examples 5 and 7

The raw materials of Part A (D-phase) and Part C (aqueous phase) of Table 5 below were mixed together and stirred at 70° C. to prepare homogeneous mixtures. The raw materials of Part B (oil phase) were mixed and stirred at 70° C., and thereby homogeneous mixtures of Part A and Part C, and Part B were prepared.

Part B was added to the mixture of Part A and Part C while stirring the mixture, and stirred using a homo mixer for 5 minutes, and thereby oil-in-water compositions of Comparative Examples 5 and 7 were prepared with the compositions shown in the following Table 5.

TABLE 5

| | | Example 1, | Comparative |
|---|---|---|---|
| | | Comparative | Examples 6 |
| | | Example 5 | and 7 |
| A | Sodium palmitoyl proline | 1 | — |
| | Sodium stearoyl glutamate | — | 1 |
| | Glycerin | 9 | 9 |
| | Butylene glycol | 3 | 3 |
| | 1,2-Hexanediol | 1.5 | 1.5 |
| B | Caprylic/capric triglyceride | 15 | 15 |
| C | Water | To 100 | To 100 |
| | Carbomer | 0.1 | 0.1 |
| | pH adjusting agent | 0.08 | 0.08 |

### Experimental Example 4—Evaluation of Emulsion Stability and Viscosity

The formulation stability over time and viscosity of the compositions of Example 1 and Comparative Examples 5 to 7, which were prepared according to Preparation Example 3, were determined in the same manner as in Experimental Example 1, and the results thereof are shown in the following Table 6. Also, to compare whether phase separation occurred or not according to the stability of the composition, the results obtained after storage at 50° C. for 8 weeks are shown in FIG. 4.

TABLE 6

| | | Comparative | Comparative | Comparative |
|---|---|---|---|---|
| | Example 1 | Example 5 | Example 6 | Example 7 |
| Three F/T cycles | stable | stable | precipitated, | precipitated, |
| | | | separated | separated |
| 50° C., 8 weeks | stable | separated | separated | separated |
| | | after 2 weeks | after 2 days | after 1 day |
| 25° C., 8 weeks | stable | separated | separated | separated |
| | | after 8 weeks | after 2 weeks | after 10 days |
| Viscosity (Day 1) | 1000 cps | 1400 cps | 1800 cps | 1800 cps |

As shown in Table 6 and FIG. 4, it can be confirmed that the composition of Example 1, including sodium palmitoyl proline and prepared by a D-phase emulsification method, exhibited both excellent formulation stability over time and low viscosity under all conditions. However, it can be confirmed that Comparative Example 5 including sodium palmitoyl proline but prepared by a general emulsification method, Comparative Example 6 prepared by a D-phase emulsification method but not including sodium palmitoyl proline, and Comparative Example 7 not including sodium palmitoyl proline and prepared by a general emulsification method all exhibited separation or precipitation over time, leading to poor emulsion stability.

1. An oil-in-water D-phase emulsion composition comprising:
N-acyl proline or a salt thereof; and
an oil.

2. The emulsion composition of claim 1, wherein the N-acyl proline or salt thereof is sodium palmitoyl proline.

3. The emulsion composition of claim 1, wherein the oil includes one or more selected from the group consisting of a silicone oil, an ester-based oil, a hydrocarbon-based oil, a triglyceride-based oil, and a mixture thereof.

4. The emulsion composition of claim 1, wherein the oil is included in an amount of 5 to 40 wt % relative to a total weight of the composition.

5. The emulsion composition of claim 1, wherein the composition has a viscosity of 4000 cps or less.

6. The emulsion composition of claim 1, wherein the composition comprises a solid emulsifier or a solid emulsification aid in an amount of 5 wt % or less relative to the total weight of the composition.

7. The emulsion composition of claim 1, wherein the composition further comprises a polyol or a polyol derivative.

8. The emulsion composition of claim 7, wherein the polyol or polyol derivative is included in an amount of 2 to 30 wt % relative to the total weight of the composition.

9. A method of preparing an oil-in-water D-phase emulsion composition, the method comprising:
preparing a D-phase including N-acyl proline or a salt thereof;
mixing the D-phase with an oil phase including an oil; and
mixing the mixture with an aqueous phase.

10. The method of claim 9, wherein the oil is included in an amount 5 to 40 wt % relative to the total weight of the composition.
